(19) United States
(12) Patent Application Publication
(10) Pub. No.: US 2018/0279918 A1
(43) Pub. Date: Oct. 4, 2018

(54) NEBULIZING DEVICES AND SYSTEMS HAVING BIOMETRIC DATA ACQUISITION AND MONITORING CAPABILITIES

(71) Applicant: SUMNER BLUFFS, LLC., Tea, SD (US)

(72) Inventors: Tony A. Hagen, Sioux Falls, SD (US); Tim Hagen, Elk Grove, CA (US); Jake Zastrow, Sioux Falls, CO (US)

(21) Appl. No.: 15/521,850

(22) PCT Filed: Oct. 20, 2015
(86) PCT No.: PCT/US2015/056505

§ 371 Date: Apr. 25, 2017

Related U.S. Application Data
(60) Provisional application No. 62/068,648, filed on Oct. 25, 2014.
(60) Provisional application No. 62/191,974, filed on Jul. 13, 2015.

(51) Int. Cl. A61B 5/117; G16H 20/10; A61M 11/00; A61K 9/00; A61B 5/00

(52) U.S. Cl. CPC A61B 5/117; G16H 20/10; A61B 5/6887; A61K 9/0019; A61M 11/005

(57) ABSTRACT

Embodiments of the present disclosure relate generally to devices and systems for administering pharmaceutical, monitored and/or biological agents via nebulization to a subject. In certain embodiments, the present disclosure provides devices, methods and systems for biometric and diagnostic data acquisition and patient monitoring before, during, and after the nebulization and administration of pharmaceutical, monitored and/or biological agents to a subject. In yet other embodiments, devices, systems and methods disclosed herein provide for delivery of agents where patients have an increased level of control over the delivery of their medication and by providing healthcare providers with meaningful and accurate biometric and diagnostic data during treatment.

### RELATED APPLICATIONS

The instant application claims the benefit of U.S. Provisional Patent Application Ser. No. 62/068,648, filed Oct. 25, 2014, and U.S. Provisional Patent Application Ser. No. 62/191,974, filed Jul. 13, 2015. These applications are incorporated herein by reference in their entirety for all purposes.

### FIELD

Embodiments of the present disclosure generally relate to devices and systems for administering pharmaceutical, biological and/or other agents via nebulization to a subject. In certain embodiments, the present disclosure provides devices, methods and systems for biometric data acquisition and monitoring before, during, and after the nebulization and administration of pharmaceutical and/or biological agents to the subject.

### BACKGROUND

The concept of personalized medicine is changing the healthcare landscape throughout the world. Personalized medicine is an emerging field that uses various diagnostic tools (e.g., genetic markers, biometric data) to help determine which medical treatments and procedures will be best for a given patient. By combining this personalized diagnostic information with a patient's medical records and individual needs, personalized medicine allows physicians and patients to develop targeted prevention and treatment plans. The goal of personalized medicine is to provide the right treatment in the right dose to the right patient at the right time.

Although great progress has been made, the goals of personalized medicine have not yet been fully realized. For example, there is a paucity of currently available drug delivery devices with the capability to administer safely and effectively one or more pharmaceutical agents to a patient. Prescription medications and over-the-counter drugs are administered to patients in various forms, and this typically requires a different device for each mode of administration. Additionally, physicians typically must not only rely on their patients to adhere to their medical instructions after leaving the clinical setting, they must also trust that their patients are accurately reporting information regarding their treatment. The ability for patients to have more control over the delivery of their medication, while at the same time providing physicians with meaningful and accurate biometric and diagnostic data during treatment would greatly augment the overall goals of personalized medicine and lead to better patient outcomes

### SUMMARY

Embodiments of the present disclosure include a pharmaceutical agent delivery and biometric data acquisition device having a housing unit, a power source, a processor and memory. Embodiments herein can also include at least one scanner operatively coupled to the processor. In accordance with these embodiments, the processor can be configured to verify the identity of a subject based upon information obtained by the at least one scanner. Other embodiments can include an accessory module interface for coupling a nebulizing unit to the device. In accordance with these embodiments, the nebulizing unit can facilitate delivery of one or more pharmaceutical agents to the subject. Other embodiments can include at least one biometric sensor for sensing biometric data of a subject/patient. In accordance with these embodiments, the biometric data can be sensed and acquired prior to, during, and/or after administering the one or more pharmaceutical agents to the subject, and the biometric data can be stored in memory. Other embodiments can include one or more data transfer ports associated with the device.

In some embodiments, the device can further include a mouthpiece and/or an image acquisition device centrally aligned with the mouthpiece. In accordance with these embodiments, the image acquisition device can include a lens, an image sensor and signal wires which operatively connect the image acquisition device to the processor as provided herein. Certain embodiments of the present invention can include at least one visual indicator associated with the device that emits at least one light signal operatively coupled to the image acquisition device and facing the same direction as the lens. Other indicators are contemplated, such as an audio indicator or the like.

In some embodiments, the device can further include at least one biometric sensor. In accordance with these embodiments, the biometric sensor can be one or more of a temperature sensor, a galvanic skin response sensor, a pulse oximeter, a carbon dioxide sensor, an oxygen sensor, an optical sensor, an air flow velocity sensor, an air pressure sensor, a chemical sensor, and a global positioning system (GPS) sensor or other known sensor.

In some embodiments, an accessory module contemplated herein can include one or more of an injectable syringe, an injectable needle, an inhaler, an inhaler canister, a syrup dispenser, a pill dispenser, a spray device, a nebulizer, a vaporizer, a misting device, and an inhalation mask.

In other embodiments, a device can further include one or more peripheral module interfaces for coupling one or more peripheral modules to the device. Peripheral modules can include one or more of a blood pressure monitor, a blood glucose monitor, a CPAP machine, an electrocardiogram device, a battery, and a battery charger. In some embodiments, the scanner includes a fingerprint reader, a pressure sensor, an ultrasonic nebulizer unit, and/or a heating coil unit. In some embodiments, the device can include an infrared (IR) transmitter and receiver, a radio-frequency identification (RFID) reader, and/or Bluetooth hardware and software components. In other embodiments, the processor further comprises one or more software programs for operating the at least one biometric sensor and for facilitating the acquisition of biometric data using the at least one biometric sensor.

Embodiments of the present disclosure can also include a system for delivering a pharmaceutical, biological or other monitored agent to and acquiring biometric data from a subject or user of the device. In accordance with these embodiments, the system can include a pharmaceutical agent delivery and biometric data acquisition device having a housing unit, a power source, a processor and memory or other agent housing unit. Embodiments herein can also include at least one scanner operatively coupled to the processor. The processor can be configured to verify the identity of a subject based upon information obtained by the at least one scanner. Other embodiments can also include at least one accessory module interface for coupling an accessory module to the device. In accordance with these embodiments, the accessory module can be configured to facilitate the delivery of one or more pharmaceutical or other agents to the subject. Other embodiment of the present invention can include at least one biometric sensor for sensing biometric data of the subject. In accordance with these embodiments, the biometric data can be sensed and acquired prior to, during, and/or after administering the one or more pharmaceutical or other agents to the subject, and the biometric data can be stored in memory. Certain embodiments can also include at least one data transfer port in the system. Other embodiments include facilitating the administration of the one or more pharmaceutical or monitored agents to the subject and acquisition of the biometric data from the subject related thereto.

Embodiments of the present disclosure can include methods for administering a pharmaceutical agent to an authorized user. In accordance with these methods, the method can include scanning a biometric identifier of a user using a pharmaceutical agent delivery and biometric data acquisition device; determining, using the biometric identifier, whether the user is approved to take a pharmaceutical agent; and administering a dose of the pharmaceutical agent from the pharmaceutical agent delivery and biometric data acquisition device, if the user is approved to take the dose of the pharmaceutical agent is approved to take the pharmaceutical agent.

In some embodiments, the method further includes use of a biometric identifier, which includes at least one of a fingerprint pattern, an iris pattern, a retina pattern, a vocal pattern, a facial-feature pattern, a pore pattern, a thermal image pattern, and a blood vessel pattern.

In some embodiments, the method can further include determining whether a user is approved to take the dose of the pharmaceutical or other monitored agent; matching the scanned biometric identifier to a stored biometric identifier, wherein the stored biometric identifier is an approved user's biometric identifier; identifying if the pharmaceutical agent is included on a list of pharmaceutical agents that are eligible to be taken by the user; identifying a recent time that the biometric identifier was scanned and the pharmaceutical agent was dispensed; and approving the administration of the pharmaceutical agent if a present time during which the biometric identifier is scanned is within an approved time period for administering the pharmaceutical or other monitored agent, based at least in part on the recent time that the biometric identifier was scanned.

In some embodiments, the method can further include recording, on the pharmaceutical agent delivery and biometric data acquisition device's memory, date and times that the pharmaceutical or other monitored agent is administered. In some embodiments, the method can further include transmitting a time that the dosage of the pharmaceutical agent was administered, the dosage amount that was administered, and an identity of the pharmaceutical agent that was administered, all to an auxiliary electronic device. In some embodiments, the method can include vaporizing the pharmaceutical or monitored agent prior to administering a dose of the pharmaceutical, biological or other monitored agent.

In some embodiments, the method can include taking an image of the user, using an image acquisition device, when administering a dosage of the pharmaceutical agent. The image acquisition device can be coupled to the pharmaceutical agent delivery and biometric data acquisition device. In some embodiments, the method can further include providing sensory feedback to the user. The sensory feedback can be at least one of visual cues, haptic feedback, and/or auditory feedback or other mode depending on the user and/or healthcare provider.

In some embodiments, the method further involves measuring a biometric response to the dose of the pharmaceutical agent. The biometric response can include at least one of a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response, and/or an exhale chemical composition response. In some embodiments, the method can further include transmitting the dose of the pharmaceutical agent and the measured biometric response to an auxiliary electronic device. In some embodiments, the method can include administering a revised dose of the pharmaceutical or other monitored agent based in part on the measured biometric response of the user.

### Definitions and Terms

As used herein, the terms “subject,” “user,” and/or “patient” can include humans and other animals or mammals that are in need of treatment and capable of using or have assisted use of devices and systems as described herein. Additionally, the terms “subject,” ^{“}user,” and/or “patient” can include humans and other mammals treated in any type of environment such as a clinical setting, non-clinical setting, experimental setting, etc.

As used herein, the terms “nebulizer,” “nebulization,” “nebulize,” and “nebulizing unit” “vaporizer” generally refer to the process or state of aerosolizing or vaporizing a mixture, solution, or suspension including one or more pharmaceutical, monitored and/or biological agents using a nebulization device to facilitate the inhalation of the pharmaceutical, monitored and/or biological agent by a subject.

As used herein the terms “pharmaceutical,” “pharmaceutical agent,” “biological agent,” “biologic,” “monitored agent,” “agent” and “drug” can mean a pharmaceutically effective compound, and/or effective compound and/or the pharmaceutically acceptable salt of a pharmaceutically effective compound, used in the treatment of a disease or condition. For example, a pharmaceutical drug or agent contemplated herein can be used in the treatment of diseases such as asthma, bronchitis, emphysema, lung infection, cystic fibrosis, alpha-1 anti-trypsin (AAT) deficiency, chronic obstructive pulmonary disease (COPD), acute respiratory distress syndrome (ARDS), infant respiratory distress syndrome (IRDS), borderline personality disorder (BPD), and macrophage activation syndrome (MAS), among many other conditions. Useful pharmaceutical agents can be delivered via inhalation, injection, ingestion, by feeding tube, and/or sublingually, according to the present disclosure, but are not limited to only those listed in the present disclosure. Generally, the agents that can be delivered using the devices and systems of the present disclosure have been approved by the U.S. Food and Drug Administration. Other agents or drugs may be used in accordance with the devices and systems of the present disclosure; the agents listed in the present disclosure are not intended to be exhaustive.

The terms “determine,” “calculate,” and “compute,” and variations thereof, as used herein, are used interchangeably and include any type of methodology, process, mathematical operation or technique.

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “computer-readable medium” as used herein refers to any storage and/or transmission medium that participate in providing instructions to a processor for execution. Such a medium is commonly tangible and non-transient and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The term “module” as used herein refers to any known or later developed hardware, software, firmware, artificial intelligence, fuzzy logic, or combination of hardware and software that is capable of performing the functionality associated with that element. Also, while the disclosure is presented in terms of exemplary embodiments, it should be appreciated that individual aspects of the disclosure can be separately claimed.

“Radio-Frequency IDentification” (RFID) refers to the use of a wireless non-contact system that uses radio-frequency electromagnetic fields to transfer data from a tag attached to an object, for the purposes of automatic identification and/or tracking. Some tags require no battery and are powered and read at short ranges via magnetic fields (electromagnetic induction) (known as passive RFID tags). Others use a local power source and emit radio waves (electromagnetic radiation at radio frequencies) (known as active RFID tags). The tag contains electronically stored information which may be read from up to several meters away. Unlike a bar code, the tag does not need to be within line of sight of the reader and may be embedded in the tracked object.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a representative block diagram of a system incorporating the pharmaceutical agent delivery and biometric data acquisition device, according to one embodiment of the present disclosure.

FIG. 2 is a representative diagram of the top view of the pharmaceutical agent delivery and biometric data acquisition device, according to one embodiment of the present disclosure.

FIG. 3 is a representative diagram of a side view of the pharmaceutical agent delivery and biometric data acquisition device, according to one embodiment of the present disclosure.

FIG. 4 is a representative diagram of the bottom view of the pharmaceutical agent delivery and biometric data acquisition device, according to one embodiment of the present disclosure.

FIG. 5 is a representative diagram of a nebulizing unit interfacing with the pharmaceutical agent delivery and biometric data acquisition device, according to one embodiment of the present disclosure.

FIG. 6 is a representative flow diagram of a method for authenticating a user using the pharmaceutical agent delivery and biometric data acquisition device, according to one embodiment of the present disclosure.

FIG. 7 is a representative flow diagram illustrating an example of methods for authenticating a user using the pharmaceutical agent delivery and biometric data acquisition device described in FIG. 6.

### DETAILED DESCRIPTION

Embodiments of the present disclosure generally relate to devices and systems for administering pharmaceutical and/or biological agents via nebulization to a subject. More specifically, the present disclosure provides devices, methods and systems for biometric data acquisition and monitoring before, during, and after the nebulization and administration of pharmaceutical and/or biological agents to a subject.

Embodiments disclosed herein can include devices having three principal components: a scanner to verify and/or authenticate a user (e.g., a fingerprint scanner), a biometric sensor to acquire user biometric data (e.g., a pulse oximeter), and a pharmaceutical delivery component (e.g., an inhalation canister) to deliver a pharmaceutical, biological or other monitored agent to the user. In accordance with these embodiments, the devices of the present disclosure can be handheld, allowing an authenticated user or caregiver to deliver a pharmaceutical or biological agent or other monitored agent while acquiring user biometric data before, during and/or after administration of the pharmaceutical or biological agent. In some embodiments, the devices of the present disclosure can also facilitate transfer of user biometric data to an authorized caregiver, health professional or physician, which can be used by the caregiver, healthcare provider or physician to evaluate accurately the user's condition and provide more effective treatment options.

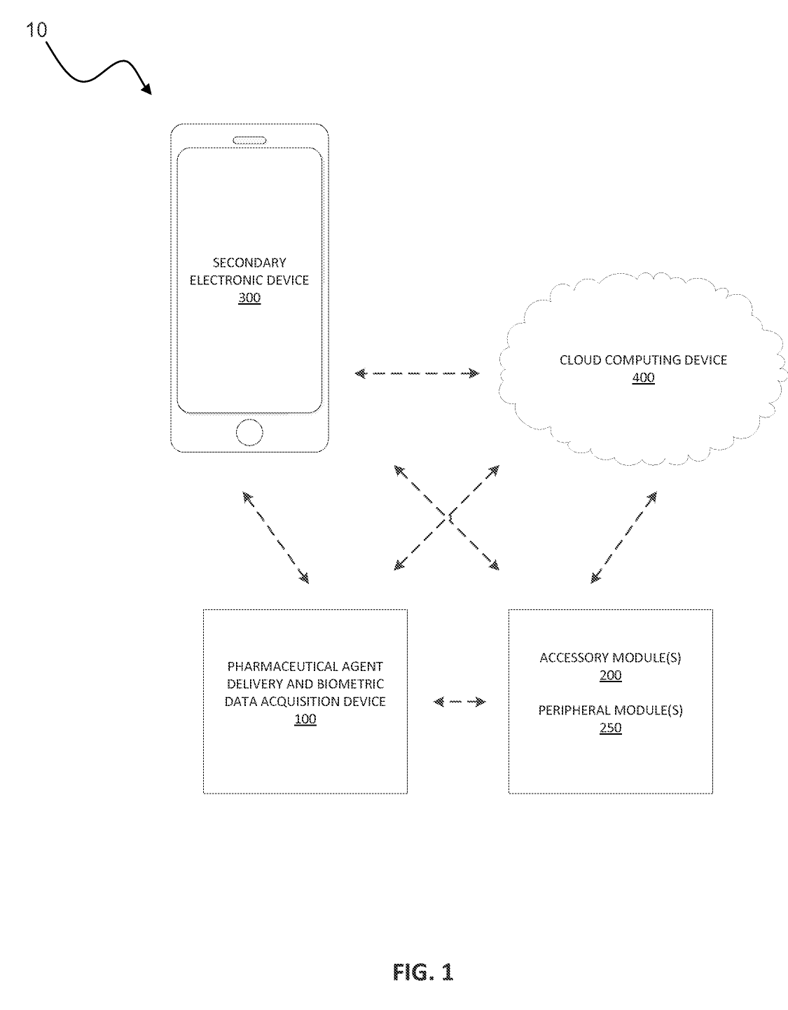

FIG. 1 is a representative block diagram of a system 10 incorporating the pharmaceutical delivery and biometric data acquisition device 100, according to one embodiment of the present disclosure. The system 10 includes a pharmaceutical agent or other agent delivery and biometric data acquisition device 100, one or more accessory modules 200, one or more peripheral modules 250, a secondary electronic device 300, and a cloud computing device 400 all of which can be communicatively coupled, using either a wired or wireless connection. However, in some embodiments, the devices 100, 200, 250, 300, 400 illustrated in FIG. 1 do not always have to be connected to one another and may only establish a connection intermittently. Furthermore, in some embodiments, the pharmaceutical agent delivery and biometric data acquisition device 100 may not connect to all the other devices 200, 250, 300, 400 illustrated in FIG. 1, but may only connect to one of the other devices 200, 250, 300, 400. For example, in some embodiments, the pharmaceutical agent delivery and biometric data acquisition device 100 may only connect to the secondary electronic device 300. In these embodiments, the secondary electronic device 300 can then connect to the cloud computing device 400. However, this is only an example and not meant to be limiting.

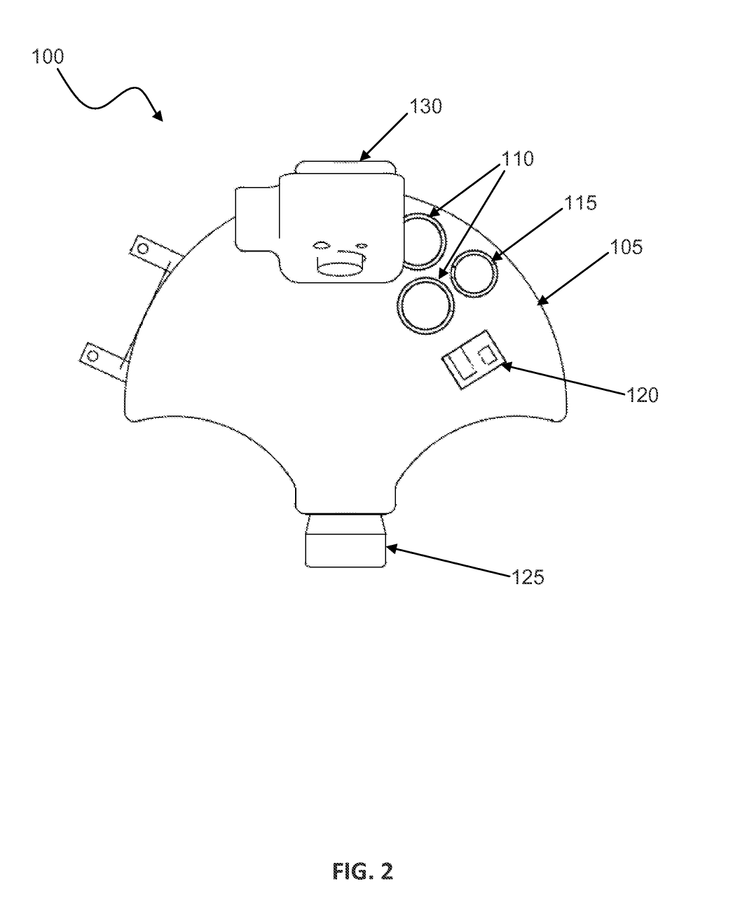

The pharmaceutical agent delivery (or other agent) and biometric data acquisition device 100, the accessory module(s) 200 and the peripheral modules(s) 250 are discussed in more detail below in FIGS. 2-4. In the illustrated example, the secondary electronic device 300 can be a smartphone. However, other exemplary secondary electronic device(s) 300 can include, but are not limited to, a telephone, a laptop computer, a tablet computer, a personal digital assistant (PDA), a digital camera or other image recording device, a gaming device, a desktop computer, a fitness tracking device, a digital display device, a docking station, or a security terminal or station. The cloud computing device 400 may be implemented, for example, as one or more servers which may be communicatively coupled to the Internet, and which may be co-located or geographically distributed.

As illustrated in FIG. 2, the pharmaceutical agent delivery and biometric data acquisition devices 100 of the present disclosure include a housing unit 105, which may be configured to contain a battery, a real time clock, and a processing device for operating a plurality of biometric sensors. The structure of the housing unit 105 may be generally configured to enable a user to grasp and operate the device without interfering with biometric data acquisition before, during, or after a pharmaceutical agent is being delivered. For example, certain devices contemplated herein can have wing-like projections facilitating grasp of the device by the user, as illustrated in FIG. 2. Other similar shapes and configurations can readily be ascertained by one of ordinary skill in the art based on the present disclosure and what is known in the art.

In some embodiments, the wing-like projections of the housing unit 105 can provide sufficient structure or surface area permitting the user to interface with various biosensors that can be included in or on the surface of the device. For example, the device can include one or more galvanic skin response sensors 110 located on the top portions of either or both of the wing-like projections of the housing unit 105 (FIG. 2). The galvanic skin response (GSR) sensors 110 of the present disclosure, can also be referred to as electrodermal response (EDR) sensors, psychogalvanic reflex (PGR) sensors, skin conductance response (SCR) sensors, or skin conductance level (SCL) sensors, generally measure electrical conductance of the skin, which can vary depending, for example, on the state of sweat or other condition of the skin. Sweating is generally considered to be controlled by the sympathetic nervous system; therefore, electrical skin conductance can provide psychological and/or physiologic biometric data about the user. In general, if the sympathetic branch of the autonomic nervous system is highly aroused, then sweat gland activity also increases, which in turn increases skin conductance. In this way, skin conductance can be used as a biometric measurement of emotional and sympathetic responses, which can be used to evaluate, for example, the efficacy and/or side effects caused by various pharmaceutical agents before, during, or after delivery of the pharmaceutical agents.

In other embodiments, the housing unit 105 can provide sufficient structure for incorporating one or more temperature sensors (FIG. 2). For example, the device can include one or more fingertip temperature sensors 115 positioned on the top portions of either of the wing-like projections of the housing unit 105 such that the temperature of a user's skin can be acquired and/or monitored before, during, and/or after a pharmaceutical agent or other agent is being delivered and/or administered. Typically, the temperature at the surface of a subject's skin changes according to blood circulation through the body tissue. The small blood vessels crossing through the tissue are surrounded by fibers of smooth muscle, which are controlled by the sympathetic nervous system. In a state of increased exertion, excitement and stress, these muscle fibers contract, causing a stenosis of vasculature. This leads to a reduction of skin temperature, because blood circulation through the tissue is reduced. In contrast, in a state of relaxation, the musculature is also bound to relax, causing the vasculature to expand. Hence, the skin temperature rises. Mental stress can lead to a lower peripheral perfusion and a decrease of skin temperature at the hands, caused by increased activity of the sympathetic nervous system. In this way, temperature of the skin at a user's fingertip can be used to as a biometric measurement for evaluating, for example, the efficacy and/or side effects caused by various pharmaceutical agents before, during, and/or after delivery of the pharmaceutical agents or other agents.

In some embodiments, the housing unit 105 can provide sufficient structure for incorporating one or more ambient temperature sensors for measuring the air temperature immediately surrounding the device, thus reflecting changes in the environmental conditions. In some embodiments, the ambient temperature sensor can be integrated with the galvanic skin response sensors and/or the fingertip temperature sensors in order to account for alterations in the environmental conditions. The integration of the various temperature sensors can provide more accurate temperature measurements of the subject for evaluating, for example, the efficacy and/or side effects caused by various pharmaceutical agents before, during, and/or after delivery of the pharmaceutical agents.

In some embodiments, a fingertip sensor can be included in the devices of the present disclosure to measure a user's heart rate. For example, a fingertip heart rate sensor unit can include an infrared light-emitting-diode (IR LED) and a photo diode, such that a user's fingertip can be placed over the sensor unit. The IR LED can transmit an infrared light into the fingertip, a part of which may be reflected back from the blood inside the finger arteries. The photo diode then senses the portion of the light that is reflected back. The intensity of reflected light depends upon the blood volume inside the fingertip, which varies every time the heart beats in accordance with changes in the amount of reflected infrared light detected by the photo diode. Other similar methods of detecting heart rate using a fingertip sensor can readily be ascertained by one of ordinary skill in the art based on the present disclosure. Monitoring a user's heart rate can be an important biometric measurement for evaluating, for example, the efficacy and/or side effects caused by various pharmaceutical agents before, during, or after delivery of the pharmaceutical agents. Other methods for measuring/detecting heart rate are known in the art and can be adapted to the device as needed.

In some embodiments, the housing unit 105 can provide sufficient structure for incorporating one or more pulse oximeters 120 (FIG. 2). For example, a pulse oximeter 120 can be used to measure the oxygen level (or oxygen saturation) in a subject's blood. Typically, the pulse oximeter 120 can be placed on a thin part of a subject's body, usually a fingertip, and two wavelengths of light are passed through the fingertip to a photodetector. The photodetector measures the changing absorbance at each of the wavelengths, allowing it to determine the absorbance due to the pulsing arterial blood alone. The pulse oximeter 120 can be used to assess a user's blood oxygenation levels and determining the effectiveness of, or need for, supplemental oxygen. The pulse oximeter 120 can also be used as a biometric measurement for evaluating, for example, the efficacy and/or side effects caused by various pharmaceutical agents before, during, and/or after delivery of the pharmaceutical agents. The pulse oximeter 120 can also be used to determine noninvasively a subject's hemoglobin level within 1-2 minutes without requiring any further equipment.

In some embodiments, the housing unit 105 can be coupled to a mouthpiece 125 that facilitates inhalation and exhalation of air from a user to the device (FIG. 2). In some embodiments, the mouthpiece 125 and the device 100 can be used for the treatment of asthma and/or asthmatic conditions, wherein for example, clenbuterol is delivered to a subject and various pulmonary biometrics are evaluated before, during, and/or after delivery of the clenbuterol in order to assess the subject's response to the clenbuterol.

In some embodiments, the mouthpiece 125 can be functionally coupled to a pulmonary function adaptor. The pulmonary function adaptor can be generally cylindrical in shape for insertion into a horizontal port in the device 100. In certain embodiments, the pulmonary function adaptor can facilitate the measurement the velocity, depth and composition of a subject's breath, measurements which are important biometrics for evaluating the health of the subject. For example, a device of the present disclosure having a pulmonary function adaptor can include one or more air pressure sensors which measure air pressure, often stated in terms of force per unit area. A pressure sensor typically acts as a transducer by generating an electrical or digital signal as a function of the pressure imposed. Sensors can be used to measure variables such as air flow, speed, and altitude. Air pressure sensors can alternatively be called pressure transducers, pressure transmitters, pressure senders, pressure indicators, piezometers and manometers, among other names, as would be appreciated by one of ordinary skill in the art based on the present disclosure and knowledge in the art.

Suitable materials that can be used to construct the housing unit 105, the mouthpiece 125, and/or the pulmonary function adaptor include, but are not limited to, various plastics and polymers materials, such as polystyrene (PS), polycarbonate (PC), acrylonitrile-butadiene-styrene (ABS), polybutylene terephthalate (PBTP), styrene acrylonitrile (SAN), polyamide (PA), polyoxymethylene (POM), polyphenylene oxide (PPO), PE, PP, PTFE and homopolymers and copolymers of these plastics and similar materials known in the art. The plastics may also be used in a filled or fiber-reinforced form, and/or coupled to portions of metals or metal alloys, such as aluminum, titanium, steel, and combinations thereof. The materials used to construct the housing unit 105 and/or the mouthpiece 125 can be surface-coated, for example with paints, varnishes or lacquers. The use of color plastics, for example colored with pigments, is also possible. In some embodiments, the housing unit 105, the mouthpiece 125, and/or the pulmonary function adaptor can be coated with substances that help to prevent contamination from microorganisms, bacteria, fungi, viruses, and the like. The coatings can be active pharmaceutical agents that reduce the growth and/or survival of these harmful microorganisms (e.g., anti-bacterial substances), or the coatings can function passively to prevent contamination, for example, by preventing adherence of these microorganism to the housing unit 105, the mouthpiece 125, and/or the pulmonary function (e.g., wetting agents).

In some embodiments, air pressure sensors can be coupled with one or more sensors designed to assess the chemical and/or gaseous composition of a user's breath. For example, the device of the present disclosure can include one or more sensors to detect carbon dioxide levels expired and/or produced by a user. A carbon dioxide sensor or CO₂ sensor typically includes infrared gas sensors (e.g., NDIR sensors) and chemical gas sensors, which can help assess the function of a subject's lungs. NDIR sensors are typically spectroscopic sensors used to detect CO₂ by its characteristic absorption. The key components include an infrared source, an interference (wavelength) filter, and an infrared detector. In some embodiments, a user breathes air through the mouthpiece 125, and the sensor measures the absorption of the characteristic wavelength of light. CO₂ sensors can also be functionally coupled with one or more air pressure sensors described above to capture a user's biometric data pertaining to both CO₂ levels and respiration rate, key biometrics used to evaluate a subject's health and disease state. Air pressure sensors and CO₂ sensors can also be used to assess, for example, the efficacy and/or side effects caused by various pharmaceutical agents before, during, or after delivery of the pharmaceutical agents. In other embodiments, sensors can be used to detect odors in a subject's breath, including an ammonia-like odor, which can be indicative of kidney failure, and/or a fruity odor, which can be indicative of ketoacidosis/diabetes and/or anorexia and other disorders.

Other sensors can also be included in the devices of certain embodiments of the present disclosure, as would be readily appreciated by one of ordinary skill in the art based on the present disclosure. For example, the devices of the present disclosure can include global positioning system (GPS) sensors, chemical sensors, thermal sensors, magnetic sensors, radiation sensors, proximity sensors, acoustic sensors, vibration sensors, acceleration sensors, moisture sensors, and the like. In some embodiments, the device can be equipped with a sensor or monitor capable of measuring a subject's blood glucose levels, as well as determining if the subject's blood glucose levels are within a certain range.

In other embodiments, a thermal imaging sensor can be included in the devices of the present disclosure to facilitate user authentication and/or as a biometric sensor. A thermal imaging sensor can be integrated with the image acquisition device to facilitate the scanning and processing of a thermal image of one or more portions of a subject's face and/or the subject's entire body. In some embodiments, the thermal imaging sensor can be used to evaluate whether the subject has a medical condition (e.g., fever) that may require immediate attention. In such embodiments, the device can be configured to send an alert message to the subject to seek immediate medical attention.

In some embodiments, a user (e.g., authenticated user, health care provider or associate of the authenticated user) can set one or more alarms using the device, such as one or more medication alarms, which can present a stimulus to the user with or without an accompanying text-based message to, for example, take one or more doses of one or more pharmaceutical or biological agents. The alarm can be a visual (e.g., flashing light) and/or auditory (e.g., ringing bell sound) stimulus that is emitted from the device. The alarm can also be pushed to another device, such as a mobile phone or computing device. In some embodiments, the alarm can take the form of an email, text message, a message from a third party mobile phone application and the like. Similarly, a user can set one or more biometric alarms, which can present a similar stimulus to the user to, for example, obtain and record one or more biometrics using the device.

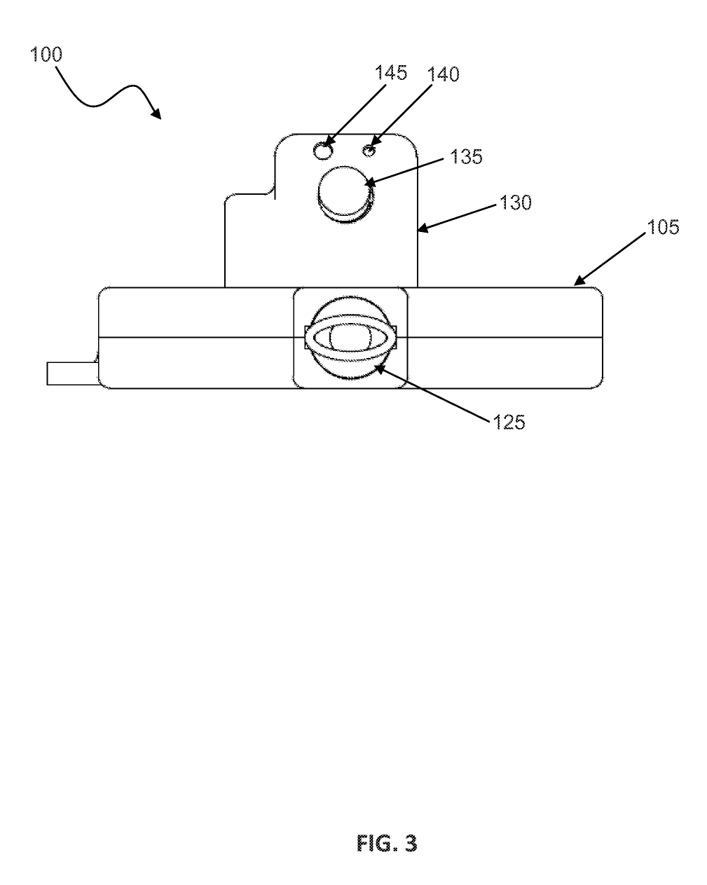

In some embodiments, devices disclosed herein can include an image acquisition device 130 (FIG. 3). The image acquisition device 130 can be generally positioned on the device such that it can be centrally aligned with the mouthpiece 125. The image acquisition device 130 comprises a lens 135, an image sensor, and signal wires which operatively connect the image acquisition device 130 to the processor in the device. In some embodiments, the image acquisition device may be a digital camera. The image acquisition device 130 can be mounted on the housing unit 105 and be electrically coupled to the processor of the device. The image acquisition device 130 generally faces the same direction as that of the mouthpiece 125, such that when a user's mouth engages the mouthpiece 125, the user's eyes will be facing the lens of the image acquisition device 130.

In some embodiments regarding a manner of operation, the image acquisition device 130 can capture a digital image and/or a series of digital images (e.g., a digital video) before, during, or after delivery of a pharmaceutical or other monitored agent. In other embodiments, the image sensor can detect a user's pupils and capture one or more images of the user's pupils before, during, and/or after delivery of a pharmaceutical agent in order to assess the efficacy and/or side effects caused by the pharmaceutical or other monitored agent. In other embodiments, the image acquisition device 130 can be used to assess the color of a user's eye, including but not limited to, the color of a user's sclera. For example, certain conditions can cause a subject's eyes to appear yellow, which can indicate dysfunction in one or more bodily organs such as the liver, gallbladder, or pancreas. Yellowing of the sclera can be used to diagnose various conditions, including alcohol abuse, hepatitis (A, B, C, D, and E), liver cancer, liver infection, and non-alcoholic fatty liver disease. In other embodiments, an image acquisition device 130 can be used to assess pupil dilation or severe reddening of an eye known to be linked to side effects of certain agents.

In other embodiments, the image acquisition device 130 can be configured to capture a digital image and/or a series of digital images that can be transferred to an auxiliary electronic device and viewed by a caregiver or health provider for diagnostic purposes. For example, the pharmaceutical agent delivery and biometric data acquisition device 100 can have an activation button functionally coupled to the image acquisition device 130 to enable a user to engage the activation button and capture a digital image or video of, for example, information pertaining to the pharmaceutical or other monitored agent (e.g., dose, lot number, etc.) or a physical manifestation of a disease condition located on the subject (e.g., wound, laceration, rash, allergic reaction, insect bite, swollen glands, etc).

In some embodiments, the image acquisition device 130 can be configured to take a picture of a subject's retina to evaluate the vascularization of the retina and/or whether the subject has a retinal vascular occlusion. A retinal vascular occlusion occurs when one of the veins or arteries carrying blood to or from the retina becomes blocked or contains a blood clot. The blockage could occur in the main vein or main artery. Blockages could also occur in the branch of veins and arteries throughout the retina. A blockage in the vein or artery of the retina can cause blood or other fluids to build up and inhibit the retina's ability to filter light properly. When light is blocked or fluids are present, sudden loss of vision can occur. The presence of a retinal vascular occlusion or blockage can be a predictor of an increased likelihood that the subject will experience a stroke or other life-threatening condition.

The pharmaceutical agent delivery and biometric data acquisition device 100 can also be equipped with a microphone that may or may not be functionally coupled to the image acquisition device 130 to facilitate real-time and/or recorded audio and/or video communication with a caregiver for diagnostic purposes.

The image acquisition device 130 can also include one or more visual indicators operatively coupled to the image acquisition device and facing the same direction as the lens 135, which emit at least one light signal. In some embodiments, the visual indicator can be an LED that emits green light 140. In other embodiments, the visual indicator can be an LED that emits white light 145. These and other visual indicators can be used to communicate directions to the user, such as when to administer a pharmaceutical agent (e.g., inhale or ingest a pharmaceutical agent). These and other visual indicators can also be used to facilitate the acquisition of biometric data from the user, such as emitting a flash of light to dilate a user's pupils. Changes in a user's pupil size or pupil dilation can be an important biometric measurement indicating, for example, the efficacy and/or side effects caused by the administration of a pharmaceutical or other monitored agent or caused by dose level of an administered agent. In accordance with these embodiments, negative visual indicators can then be used to adjust, change or eliminate use of the agent for the user. Additionally, the device can be configured to send instructions to a user to activate an eye tracking program that uses visual stimulation, such as pulses of light, to assess various neurological problems, including brain diseases and brain injuries (e.g., concussions). Eye tracking technology and testing protocols are well established and can obtain hundreds of data points during, for example, a 30-second test facilitated by the video recording capability of the image acquisition device 130.

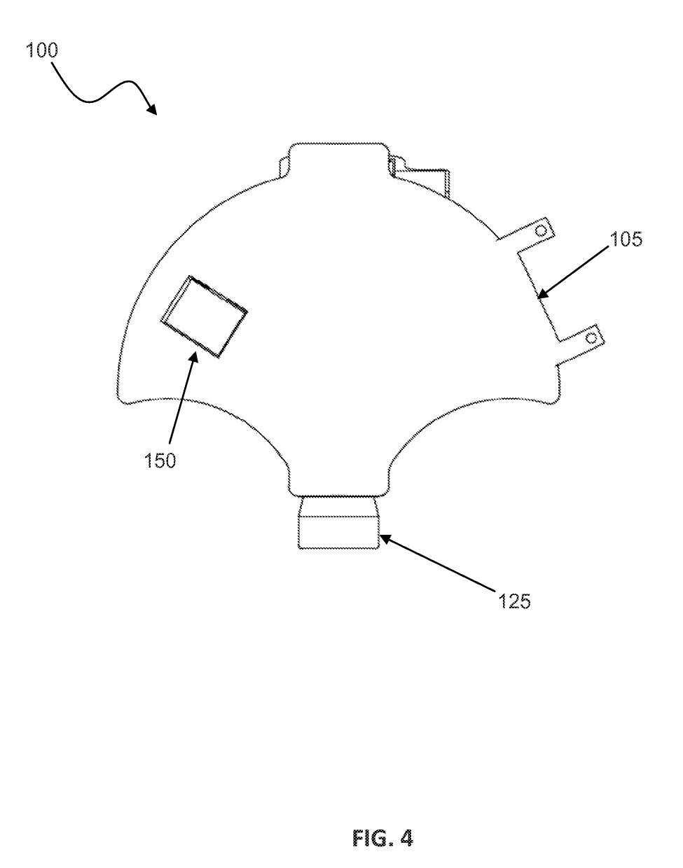

Some embodiments disclosed herein can include one or more scanners associated with the device which authenticate and/or verify the identity of a user or caregiver that will be administering a pharmaceutical or other monitored agent to a subject. In some embodiments, images captured using the image acquisition device 130 can be used for retinal scanning and/or facial recognition to prevent unauthorized users from being able to take a pharmaceutical agent meant for the imprinted user and/or tampering with the device. In other embodiments, the device of the present disclosure can include a fingerprint scanner 150 to prevent unauthorized users from administering a pharmaceutical agent and/or tampering with the device (FIG. 4). The device of the present disclosure can store in its memory a plurality of distinct user fingerprints, (e.g., biometric identifiers), and the device can be programmed to correlate a particular fingerprint with certain device settings for a particular user. In this way, the device of the present disclosure can be used by more than one user, if desired, for example, a family of users, without the need for multiple devices for each person in need thereof or for each pharmaceutical or monitored agent being administered. In other aspects, the device can be configured to be accessed specifically by an authorized user such as a nurse, health provider, parent or other caregiver, and the nurse, health provider, parent or caregiver's fingerprint or other biometric identifier can be used to access the patient's settings on the device, as the patient may need to be restricted from using the device on his/her own or the patient may not be capable of using the device without supervision or aide (e.g., a child or elderly person). The fingerprint scanner 150 can also be used in conjunction with a lockout mechanism in which the device will be “locked out” or inactive for a given operation of a particular delivery program if the user's fingerprint is not recognized.

Some embodiments of the present disclosure can include memory in electrical communication with the processor of the device and configured to facilitate the acquisition and storage of biometric data acquired using various biometric sensors from one or more users. Biometric data can include, but is not limited to, images, air flow rates, air composition, fingerprints, oxygen levels, carbon dioxide levels, skin electrical conductance measurements, time, temperature, heat, user identification, dosages, usage rates, medication batch numbers, bar codes, and any other biometric data that can be captured using the various biometric sensors of the present disclosure. User biometric data can be stored and uploaded/downloaded wirelessly to a variety of memory storage and data processing devices, including but not limited to, cell phones, smart phones, watches, computers, laptops, tablets, servers, and the like. User biometric data can also be stored and uploaded/downloaded via a wire or cable to a variety of other memory storage and data processing devices, including but not limited to, cell phones, smart phones, watches, computers, laptops, tablets, servers, and the like. In such embodiments, the device can have one or more data transfer ports. The ability to acquire and store a subject's biometric data over time provides physicians with more accurate diagnostic and biometric data with which to evaluate the subject, and allows for more general patient trends to be analyzed with relation to, for example, a specific disease indication.

Some embodiments of the present disclosure can include one or more accessory module interfaces that facilitate the functional coupling of one or more accessory modules 200 to the device. Examples of accessory modules 200 include, but are not limited to, an injectable syringe, an injectable needle, an inhaler, an inhaler canister, a syrup dispenser, a pill dispenser, a spray device, a nebulizer, a vaporizer, a misting device, an inhalation mask, and the like. The accessory module interfaces allow for one or more accessory modules 200 to be coupled to the device such that one or more pharmaceutical agents can be administered to a user.

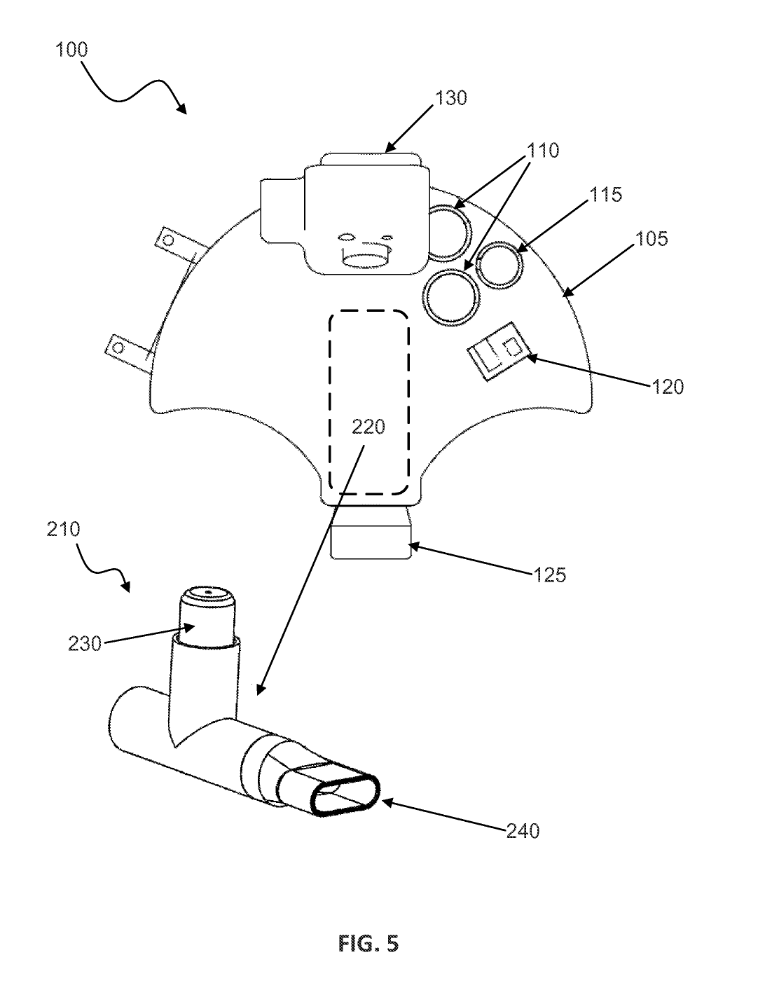

Some embodiments of the present disclosure can include an accessory module that includes a nebulization unit 210 (see for example, FIG. 5). The process of nebulizing a substance generally involves phenomenon known as cavitation, which is the formation of vapor cavities in a liquid (e.g., voids) that are the consequence of cavitational forces acting upon the cavitational liquid. For example, ultrasonic nebulization uses the process of cavitation to create a mist using ultrasonic waves propagating through a liquid and impinging on a liquid-gas interface. Heat-based nebulizers generally include a heating element or heating coil that raises the temperature of a liquid to a sufficient degree to vaporize the liquid. Other nebulization mechanisms can also be used with the devices of the present disclosure, including but not limited to, electrical mechanisms, piezoelectric mechanisms, jet nebulization mechanisms (e.g., atomizers), and vibrating mechanisms, and the like. In some embodiments, the specific nebulization mechanism used may depend at least in part on the viscosity of the liquid being vaporized, with more viscous liquids generally requiring heat-based nebulizing mechanisms.

As illustrated in FIG. 5, embodiments herein regarding a nebulizing unit 210 can functionally couple to the pharmaceutical agent delivery and biometric data acquisition device 100 via an accessory module interface 220. An ultrasonic element and/or the heating element (e.g., vaporizing elements) can be included within the nebulizing unit 210 itself, along with the liquid pharmaceutical agent contained within the reservoir 230. In some embodiments, the nebulizing unit 210 vaporizes a liquid pharmaceutical agent (e.g., albuterol, medically prescribed cannabis oil, etc.) contained in the reservoir 230 by an ultrasonic mechanism, and in other embodiments, the nebulizing unit 210 vaporizes a liquid pharmaceutical agent contained in the reservoir 230 by a heat-based mechanism. The nebulizing unit 210 can be coupled to the pharmaceutical agent delivery and biometric data acquisition device 100 such that the mouthpiece 125 of the device 100 aligns with the pharmaceutical agent exit port 230 of the nebulizing unit 210 to facilitate the inhalation of the pharmaceutical agent by a subject. In certain embodiments, a subject can activate the nebulizing unit 210 via an activation mechanism contained within the nebulizing unit 210, or via an activation mechanism contained within the pharmaceutical agent delivery and biometric data acquisition device 100.

In some embodiments, nebulizing unit 210 may be coupled to a facemask (e.g., an oxygen mask) or some other auxiliary breathing accessory to facilitate the inhalation of the vaporized liquid pharmaceutical agent. In some embodiments, the mask may be configured to be used by a child so that the child is not required to administer the vaporized liquid pharmaceutical agent, but simply inhales the prescribed pharmaceutical agent. The use of a facemask or other auxiliary breathing accessory also maximizes the amount of the vaporized pharmaceutical agent inhaled by the subject or patient.

In some embodiments, the vaporizing element of the nebulizing unit 210 may be electrically coupled to the processor of the device 100, such that the user can activate the nebulizing unit 210 in conjunction with the activation of one or more biosensors to facilitate the acquisition of biometric data using the biosensors before, during, and/or after administration of a pharmaceutical agent using the nebulizing unit 210. In other embodiments, the heating element can produce temperatures, for example, between 300° F. and 500° F. Additionally or alternatively, the ultrasonic element can emit ultrasonic frequencies to vaporize a pharmaceutical agent. When, for example, a liquid pharmaceutical agent is brought in contact with or adjacent to the vaporizing element, the fluid becomes vaporized, and the fluid vapors can be inhaled by a user.

In other embodiments, the pharmaceutical agent can be contained within a sealed container having a tamper resistant construction (e.g., a canister or inhaler used to deliver clenbuterol). In other embodiments, the pharmaceutical agent can be contained within a sterile syringe dispensing device or mister (e.g., insulin). In certain embodiments, accessory module interfaces can be configured to allow a variety of such modules to be coupled to the device, such that the device can facilitate the administration of the pharmaceutical agent to a user. For example, the device can include an actuator mounted on the housing unit 105 and electrically coupled to the processor of the device. The actuator can be configured to, for example, activate a vaporizing element to vaporize a pharmaceutical agent. The actuator can also be coupled to a biosensor, such as a fingerprint scanner, to allow vaporization of a pharmaceutical agent only when the fingerprint of an authorized user is detected.

In certain aspects, the device can include a mechanism for monitoring amount or dosage of a pharmaceutical or other monitored agent administered to a subject or user. For example, the device can include an IR transmitter and receiver which can be used to evaluate the distance a syringe dispenser has travelled in relation to a starting point, which can correspond to a single dose of a pharmaceutical agent. Additionally, the device can include a radio-frequency identification (RFID) reader, which can be used to assess the batch, date, amount and source of a particular pharmaceutical or other monitored agent.

In some embodiments, peripheral accessory modules or peripheral modules can be functionally coupled to the device of the present disclosure via peripheral module interfaces rather than accessory module interfaces. In certain devices, a peripheral module requires its own power source separate from the device, which can preclude the peripheral module from being coupled to the device via an accessory module interface. The peripheral accessory interface can be a port, including any electronic data transfer port, such as a USB port, a firewire port, and the like. Peripheral modules can include, for example, blood pressure monitors, blood glucose monitors, CPAP machines, and/or electrocardiogram machines, as well as peripheral modules for providing additional power to the device, such as a battery or a battery charging device, and devices that enable the use of Bluetooth™ and Wi-Fi™ compatibility. As with accessory modules, some peripheral modules can be functionally coupled to the processor of the device of the present disclosure to facilitate the delivery of a pharmaceutical or other monitored agent and/or the acquisition of biometric data from a user.

In other embodiments, the device can be coupled to a CPAP machine (Continuous Positive Airway Pressure) or a baby monitor (e.g., monitors used to assess Sudden Infant Death Syndrome, or SIDS), or other such medical monitoring peripheral devices. The device can be used to acquire further biometric data that may not possible using the medical monitoring peripheral device, and/or the device can be used to integrate the biometric data acquired using the medical monitoring peripheral device. In other embodiments, the device can be coupled to a motor vehicle, such that operation of the motor vehicle (e.g., starting a car) by the subject will only be allowed if certain biometric parameters are met. This feature can help prevent a subject who is taking various pharmaceutical and biological agents from operating a motor vehicle while impaired.

In some embodiments, a peripheral module can be a secondary electronic device, such as a docking station. The docking station can be used to charge the device, and can include various other accessory ports, such as an Ethernet port and/or a communication port to support a telephone landline. Additionally, the docking station can be configured to sterilize the device between uses and/or between uses by multiple users to minimize and/or prevent bacterial, fungal, and viral contamination. For example, the docking station can be configured to contain one or more sources of UV light to reduce contamination when the device is housed in the docking station. The docking station can also be configured to combine the sterilization power of UV light, purifying hydroxyl and/or activated oxygen radicals, and photo-ionization to purify the internal and external components of the device. To facilitate this sterilization process, the docking station can be equipped with various air flow mechanisms, which assist with both the activation and circulation of the hydroxyl and oxygen radicals through the device. These and other sterilization mechanisms can be included in the docking station, as would be readily recognized by one of ordinary skill in the art based on the present disclosure.

Various pharmaceutical and biological agents can be administered to a subject using the devices, systems, and methods of the present disclosure. These pharmaceutical, biological and other monitored agents can include, but are not limited to, those approved by the U.S. Food and Drug Administration, such as, for example, albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

Pharmaceutical, biological or other agents that can be administered to a subject using the devices, systems, and methods of the present disclosure include, but are not limited to, those agents that have not yet been approved by the U.S. Food and Drug Administration but are known to be of use to treat a disease or a condition, such as, for example, 13-cis-retinoic acid, 2-pentenylpenicillin, L-alphaacetylmethadol, S-adenosylmethionine, acebutolol, aceclofenac, acetaminophen, acetaphenazine, acetophenazine, ademetionine, adinazolam, adrafinil, ahnotriptan, albuterol, albuterol, albuterol sulfate, alfentanil, alfentanil HCl, alizapride, allylprodine, alminoprofen, almotriptan, alperopride, alphaprodine, alpidem, alseroxion, amantadine, ambrisentan, amesergide, amfenac, aminopropylon, amiodarone HCl, amisulpride, amitriptyline, amixetrine, amlodipine, amoxapine, amoxicillin, amperozide, amphenidone, amphetamine, ampicillin, amylpenicillin, andropinirole, anileridine, apazone, apomorphine, apomorphinediacetate, atenolol, atropine sulfate, azacyclonol, azasetron, azatadine, azidocillin, bacille Calmette-Guerin, baclofen, beclomethasone dipropionate, benactyzine, benmoxine, benoxaprofen, benperidol, benserazide, benzpiperylon, benzquinamide, benztropine, benzydramine, benzylmorphine, benzylpenicillin, bezitramide, binedaline, biperiden, bitolterol, bitolterol mesylate, brofaromine, bromfenac, bromisovalum, bromocriptine, bromopride, bromperidol, brompheniramine, brucine, buclizine, budesonide, budesonide; formoterol fumarate, budipine, bufexamac, buprenorphine, bupropion, buramate, buspirone, butaclamol, butaperazine, butorphanol, butriptyline, cabergoline, caffeine, calcium-N-carboamoylaspartate, cannabinoids, Cannabis, Cannabis oil, captodiamine, capuride, carbamazepine, carbcloral, carbenicillin, carbidopa, carbiphene, carbromal, carfecillin, carindacillin, caroxazone, carphenazine, carpipramine, carprofen, cefazolin, cefinetazole, cefinetazole, cefoxitin, cephacetrile, cephalexin, cephaloglycin, cephaloridine, cephalosporin C, cephalosporins, cephalotin, cephamycin A, cephamycin B, cephamycin C, cephamycins, cepharin, cephradine, cericlamine, cetrizine, chloralbetaine, chlordiazepoxide, chlorobutinpenicillin, chlorpheniramine, chlorpromazine, chlorprothixene, choline, cialis, cilazaprol, cilostazol, cinchophen, cinmetacin, cinnarizine, cipramadol, citalopram, clebopride, clemastine, clobenzepam, clocapramine, clomacran, clometacin, clometocillin, clomipramine, clonidine, clonitazene, clonixin, clopenthixol, clopriac, clospirazine, clothiapine, clovoxamine, cloxacillin, clozapine, codeine, cotinine, cromolyn sodium, cyamemazine, cyclacillin, cyclizine, cyclobenzaprine, cyclosporin A, cyproheptadine, deprenyl, desflurane, desipramine, dexamethasone sodium phosphate, dexfenfluramine, dexmedetomidine, dextroamphetamine, dextromoramide, dextropropoxyphene, diamorphine, diazepam, diclofenac, dicloxacillin, dihydrocodeine, dihydroergokryptine, dihydroergotamine, diltiazem, diphenhydramine, diphenicillin, diphenidol, diphenoxylate, dipipanone, disulfiram, dolasetronmethanesulfonate, domeridone, dornase alfa, dosulepin, doxepin, doxorubicin, doxylamine, dronabinol, droperidol, droprenilamin HCl, duloxetine, eletriptan, eliprodil, enalapril, enciprazine, enflurane, entacapone, entonox, ephedrine, epinephrine, eptastigmine, ergolinepramipexole, ergotamine, ergotamine tartrate, etamiphyllin, etaqualone, ethambutol, ethoheptazine, etodolac, famotidine, fenfluramine, fentanyl, fexofenadine, fientanyl, flesinoxan, fluconazole, flunisolide, fluoxetine, flupenthixol, fluphenazine, flupirtine, flurazepam, fluspirilene, fluticasone propionate, fluvoxamine, formoterol fumarate, frovatriptan, gabapentin, galanthamine, gepirone, ghrelin, glutathione, granisetron, haloperidol, halothane, heliox, heptylpenicillin, hetacillin, hydromorphone, hydroxyzine, hyoscine, ibuprofen, idazoxan, iloprost, imipramine, indoprofen, insulin (recombinant human), ipratropium bromide, iproniazid, ipsapiraone, isocarboxazid, isoetharine hydrochloride, isoflurane, isometheptene, isoniazid, rifampin, pyrazinamide, ethambutol, isoproterenol, isoproterenol hydrochloride, isoproterenol bitartrate, isosorbide dinitrate, ketamine, ketoprofen, ketorolac, ketotifen, kitanserin, lazabemide, leptin, lesopitron, levalbuterol hydrochloride, levodopa, levorphanol, lidocaine, lisinopril, lisuride, lofentanil, lofepramine, lomustine, loprazolam, loratidine, lorezepam, loxapine, maprotoline, mazindol, mazipredone, meclofenamate, mecloqualone, medetomidine, medifoxamine, melperone, memantine, menthol, meperidine, meperidine HCl, meptazinol, mesoridazine, metampicillin, metaproterenol, metaproterenol sulfate, methacholine chloride, methadone, methaqualone, methicillin, methprylon, methsuximide, methyphenidate, methyprylon, methysergide, metoclopramide, metofenazate, metomidate, metopimazine, metopon, metoprolol, metralindole, mianserin, midazolam, milnacipran, minaprine, mirtazapine, moclobemide, mofegiline, molindrone, mometasone furoate, morphine, nabilone, nadolol, nafcillin, nalbuphine, nalmefene, nalorphine, naloxone, naltrexone, naratriptan, nedocromil, sodium, nefazodone, nefopam, nicergoline, nicotine, nicotine, nifedipine, nisoxetine, nitrous oxide, nitroglycerin, nomifensine, nortriptyline, obestatin, olanzapine, omoconazole, ondansetron, orphenadrine, oxprenolol, oxycodone, palonosetron, papaveretum, papaverine, paroxetine, pemoline, penfluridol, penicillin N, penicillin O, penicillin S, penicillin V, pentamidine isethionate, pentazocine, pentetate, calcium trisodium, pentetate, zinc trisodium, pentobarbital, peptides, pergolike, pericyazine, perphenazine, pethidine, phenazocine, pheneizine, phenobarbital, phentermine, phentolamine, phenyhydrazine, phosphodiesterase-5, pilocarpine, pimozide, pipamerone, piperacetazine, pipotiazine, pirbuterol acetate, pirbuterolnaloxone, piroxicam, pirprofen, pizotifen, pizotyline, polyeptides, polypeptide YY, pramipexole, prentoxapylline, procaine, procaterol HCl, prochlorperazine, procyclidine, promazine, promethazine, propacetamol, propanolol, propentofylline, propofol, propoxyphene, propranolol, proteins, protriptyline, quetiapine, quinine, rasagiline, reboxetine, remacemide, remifentanil, remoxipride, retinol, ribavirin, rimonabant, risperidone, ritanserin, ritodrine, rizatriptan, roxindole, salicylate, salmeterol xinafoate, salmetrol, scopolamine, selegiline, sertindole, sertraline, sevoflurane, sibutramine, sildenafil, spheramine, spiperone, sufentanil, sulpiride, sumatriptan, tandospirone, terbutaline, terguride, testosterone, testosterone acetate, estosterone enanthate, testosterone proprionate, tetrahydrocannabinol, thioridazine, thiothixene, tiagabine, tianeptine, timolol, tiotropium bromide monohydrate, tizanidine, tobramycin, tofenacin, tolcapone, tolfenamate, tolfenamicacid, topiramate, tramadol, tranylcypromine, trazadone, triamcinolone acetonide, triethylperazine, trifluoperazine, trifluperidol, triflupromazine, trihexyphenidyl, trimeprazine, trimethobenzamide, trimipramine, tropisetron, tryptophan, valproicacid, vardenafil, venlafaxine, verapamil, vigabatrin, viloxazine, yohimbine, zafirlukast, zalospirone, zanamivir, zileuton, ziprasidone, zolmitriptan, zolpidem, zopiclone, zotepine, zuclopenthixol, and combinations and derivatives thereof.

In some embodiments, the pharmaceutical agent delivery and biometric data acquisition devices of the present disclosure can be used to administer unapproved drugs, pre-approved drugs, and/or drugs subject to clinical trials. For example, the devices can be used to assess the efficacy of various pharmaceutical and biological agents that are being evaluated in the context of a clinical trial. Test subjects can use the device in conjunction with clinical research being conducted to evaluate a drug's ability to attain or not attain certain clinical outcomes. The device can facilitate the acquisition of biometric data from the test subjects, as well as the aggregation of that data, in an effort to evaluate whether an experimental drug has therapeutic potential.

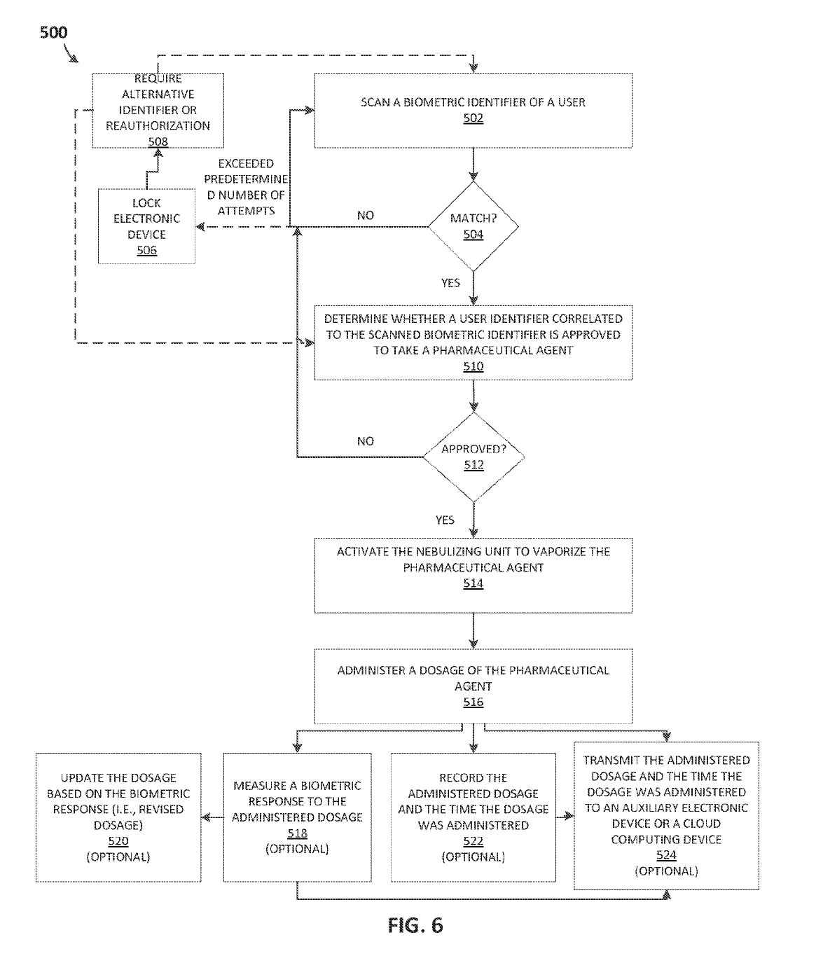

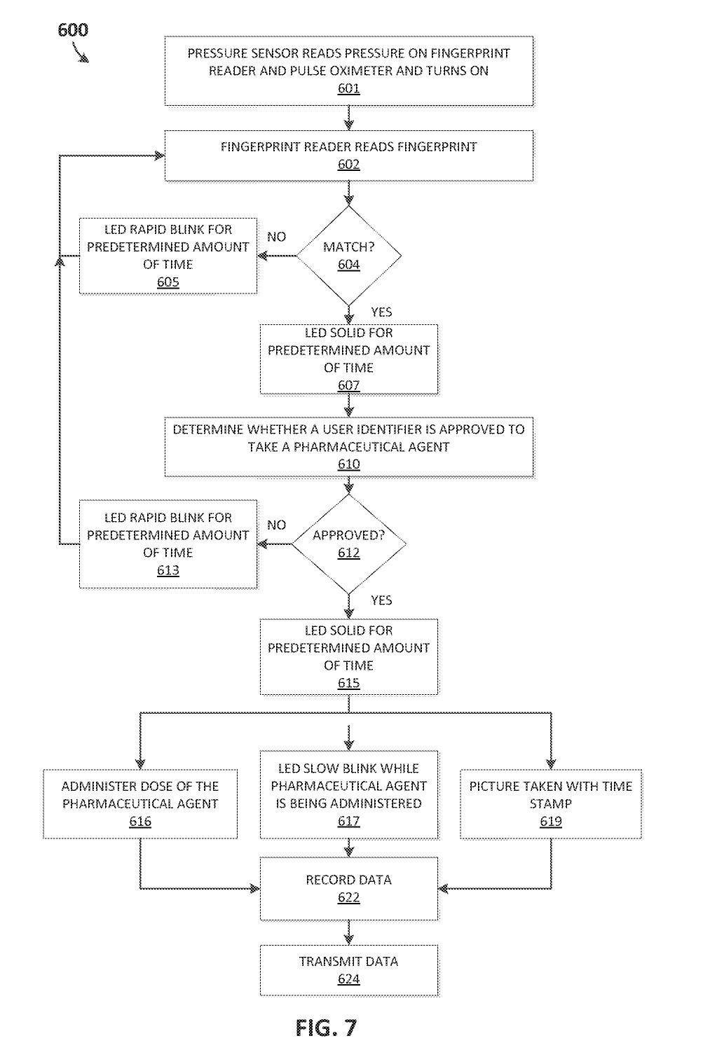

FIG. 6 is an illustration of a flow diagram of an exemplary method 500 for administering a pharmaceutical or biological agent. In some embodiments, the pharmaceutical agent delivery and biometric data acquisition device discussed throughout method 500 can have some or all of the same characteristics as the pharmaceutical agent delivery and biometric data acquisition device 100 described above in FIGS. 1-4. The pharmaceutical agent delivery and biometric data acquisition device will also be referred to herein as the “biometric data acquisition device.” For example, the pharmaceutical agent delivery and biometric data acquisition device can be turned on by holding a button (e.g., a fingerprint reader and/or pulse oximeter incorporated into the pharmaceutical agent delivery and biometric data acquisition device) for a predetermined amount of time, or by blowing air into or sucking air through the device. As another example, the pharmaceutical agent delivery and biometric data acquisition device can provide sensory feedback to a user intermittently during method 500. Examples of sensory feedback include, but are not limited to: visual cues, haptic feedback, or auditory feedback. As another example, the pharmaceutical agent delivery and biometric data acquisition device can take an image of the user intermittently during method 500. An example of sensory feedback is discussed in more detail in relation to FIG. 7 below. As another example, the pharmaceutical agent delivery and biometric data acquisition device can have wired and wireless connectivity. As another example, the pharmaceutical agent delivery and biometric data acquisition device can measure biometric responses of a user. This list, however, is not inclusive and, therefore, not meant to be limiting.

Method 500 begins by sensing a biometric identifier of a user using a pharmaceutical agent delivery and biometric data acquisition device (block 502). In exemplary embodiments, the biometric identifier includes, but is not limited to, the following: a fingerprint pattern, an iris pattern, a retina pattern, a vocal pattern, a facial-feature pattern, a pore pattern, a thermal image pattern, and a blood vessel pattern. The biometric data acquisition device can be equipped with various sensors and software to measure one or more of these biometric identifiers, as described above. In some embodiments, method 500 can begin when one or more audio sensors detects one or more audio signals from an authorized user, including the patient himself, or an authorized caregiver.

At block 504, a determination can be made whether the scanned biometric identifier matches a stored biometric identifier. The stored biometric identifier can be an approved user's biometric identifier. In some exemplary embodiments, a stored biometric identifier can be securely stored in the pharmaceutical agent delivery and biometric data acquisition device's memory. Furthermore, in some exemplary embodiments, a stored biometric identifier can be concurrently securely stored on an auxiliary electronic device (e.g., a smartphone or a cloud computing device) that the pharmaceutical agent delivery and biometric data acquisition device can connect to, either wired or wirelessly. Or, in some other exemplary embodiments, the stored biometric identifier may not store in the pharmaceutical agent delivery and biometric data acquisition device's memory, but only stored on an auxiliary electronic device, which the device can connect to, either wired or wirelessly. In exemplary embodiments, the stored biometric identifier can be included in a secure database of stored biometric identifiers. In exemplary embodiments, biometric identifiers for more than one user can be stored in the secure database of biometric identifiers and more than one biometric identifier for each user can be stored in the secure database of biometric identifiers.

In order to populate a list of stored biometric identifiers, an enrollment process can be undertaken. The enrollment process may include determining what biometric identifiers are to be used in method 500, enrolling each of those biometric identifiers using an iterative process, so that a fingerprint pattern, retina pattern, etc. can be recognized from various angles and under different conditions, and storing the enrollment data in the memory of the pharmaceutical agent delivery and biometric data acquisition device, or an auxiliary electronic device.

If the scanned biometric identifier matches a stored biometric identifier at block 504, then the method 500 continues to block 510. If the scanned biometric identifier does not match a stored biometric identifier, then method 500 can proceed back to scanning the biometric identifier at block 502. However, in some exemplary embodiments, if method 500 proceeds to scan a biometric identifier a predetermined number of times but is unable to match the scanned biometric identifier to a stored biometric identifier, then method 500 can proceed to locking the biometric data acquisition device at block 506. The predetermined number of times can be configurable when setting up the biometric data acquisition device. In some exemplary embodiments, method 500 will try to match the scanned biometric identifier to a stored biometric identifier three times before locking the biometric data acquisition device. In some other exemplary embodiments, method 500 will attempt to match the scanned biometric identifier to a stored biometric identifier five times before locking the biometric data acquisition device. In yet other exemplary embodiments, method 500 will attempt to match the scanned biometric identifier to a stored biometric identifier an unlimited number of times without locking the biometric data acquisition device.

In the embodiments disclosed herein where method 500 proceeds to block 506 and locks the biometric data acquisition device, method 500 can proceed to block 508 and require either an alternate identifier or reauthorization to unlock the pharmaceutical agent delivery and biometric data acquisition device. In some exemplary embodiments where method 500 requires an alternative identifier at block 508, a different biometric identifier than the one previously scanned may be scanned and matched to a stored biometric identifier in order to unlock the biometric data acquisition device. For example, if the biometric identifier initially scanned by the biometric data acquisition device was a fingerprint, then a user's retina may be scanned and matched to a stored biometric identifier in order to unlock the biometric data acquisition device. Or, as another exemplary embodiment, a passcode may be entered into the biometric data acquisition device in order to unlock the biometric data acquisition device. In other embodiments, block 508 may require reauthorization of the biometric data acquisition device by the manufacturer of the device, a certified healthcare professional or other third-party.

Once the biometric data acquisition device is unlocked, in some embodiments, method 500 can proceed back to block 502 or to block 510, depending on how the biometric data acquisition device was unlocked. For example, if a passcode was entered, method 500 may proceed back to 502 to identify a biometric identifier of a user since a biometric identifier was never matched to a stored biometric identifier. As another example, if a retina was scanned and the retina pattern is matched to a stored biometric identifier to unlock the biometric data acquisition device, method 500 may proceed to block 510 since a biometric identifier was matched to a stored biometric identifier. In yet another example, if the biometric data acquisition device was unlocked by the manufacturer, a healthcare professional or other third-party, the person or entity that unlocked the biometric data acquisition device may determine whether method 500 proceeds to block 502 or to block 510.

In methods disclosed herein where the scanned biometric identifier matches a stored biometric identifier at block 504, then method 500 determines, using the biometric identifier, whether the user is approved to take a pharmaceutical agent of a list of approved pharmaceutical agents for example, at block 510. In certain embodiments, determining whether the user is approved to take a pharmaceutical agent can include matching the scanned biometric identifier to a stored biometric identifier, wherein the stored biometric identifier can be an approved user's biometric identifier. Additionally, in exemplary embodiments, each stored biometric identifier can be correlated to a user identifier of the user. The user identifier may be the name of the user, the social security number of the user or rendition thereof, a username of the user, or a random number assigned to the user during configuration of the pharmaceutical agent delivery and biometric data acquisition device. A random number or username may be used to protect the privacy of the user, in addition to the biometric identifier being correlated to a user identifier.

In addition to being correlated to a biometric identifier, the user can be linked to a list of pharmaceutical agents that are eligible to be taken by the user based on for example, medical history of the user. In exemplary embodiments, the list of pharmaceutical agents may include all the pharmaceutical agents currently and previously prescribed to the user of the user identifier. If the user has never been prescribed a pharmaceutical agent, the list of prescribed pharmaceutical agents can be the null set. In some embodiments, the list of prescribed pharmaceutical agents can be uploaded to the device by a healthcare professional. This can be done either remotely when the biometric data acquisition device is either wired or wirelessly connected to a network or when the biometric data acquisition device is in the presence of a healthcare professional. In some exemplary embodiments, the list of pharmaceutical agents may include over-the-counter pharmaceuticals, nutraceuticals, minerals, supplements, vitamins, and the like.

In addition to correlating a potential list of pharmaceutical agents that are available for use by the user (prescribed, monitored and non-prescribed) for a particular purpose or in general, determining whether the user affiliated with the user identifier is approved to take a target pharmaceutical agent may include determining the present time (realtime) and date during which the biometric identifier is scanned, when the last time or any previous time a biometric identifier was scanned or when the pharmaceutical agent was administered to the user and based in part on this information, whether the instant time and/or date is within a time period that the pharmaceutical agent is allowed, safe or optimal to be taken.

If a user is approved to take a pharmaceutical agent, then the biometric data acquisition device can give sensory feedback (e.g. visual or audio signal) to the user of the user identifier that the user is approved to take a pharmaceutical agent. In some exemplary embodiments, to determine whether a specific or family of pharmaceutical agents or monitored agents are approved to be taken by the user of the user identifier, the agent may be associated with the biometric data acquisition device by an authenticated user or an approved caregiver or healthcare provider. Then, the pharmaceutical agent delivery and biometric data acquisition device can determine whether the coupled pharmaceutical agent is approved to be taken by the user. The pharmaceutical agent delivery and biometric data acquisition device can determine what pharmaceutical agent is associated with the biometric data acquisition device. In accordance with these embodiments, this can be determined in a variety of ways including, but not limited to, radio-frequency identification (RFID), resistance sensing, barcode scanning, etc. In some other exemplary embodiments, to determine whether a specific pharmaceutical agent is approved to be taken by the user, the pharmaceutical agent delivery and biometric data acquisition device can include an input and sensory feedback device for selecting a specific or family of pharmaceutical agents from a list of pharmaceutical agents. Similar to blocks 502-510, sensory feedback can be given to the user throughout the process of determining whether a user is approved to take a pharmaceutical agent.

In certain embodiments, stored biometric information can be used to determine whether one or more of a subject's current biometrics is anomalous or abnormal and whether this observation can be connected to administration of a particular pharmaceutical, monitored or biological agent. For example, a subject's biometric data can be acquired and stored on the device or an auxiliary electronic device. If a subject's specific instantaneous biometric response is outside a certain usage range, which has been established by the subject's recent history of biometric responses aggregated together, an alarm may be triggered by the device, even if the biometric response has been determined to be within an acceptable, previously determined range (e.g., a clinical range determined from patient trials). In this way, the device can be customized to operate according to a subject's individualized biometric responses.

At block 512, for example, if the user identifier is approved to take a pharmaceutical agent of interest to treat a disease or condition, method 500 may proceed to block 514 or block 516. On the other hand, if the user identifier is not approved to take the pharmaceutical agent of interest, then method 500 may proceed to block 502 or the method 500 ends. Similar to the blocks above, sensory feedback can be given to the user as to whether method 500 is proceeding to block 502, block 514, block 516, or method 500 is ending. Depending on the feedback, a user where the pharmaceutical agent is not approved can receive feedback to contact their physician or seek alternative assistance.

In some embodiments, method 500 proceeds to block 514 where the approved pharmaceutical agent can be vaporized by the pharmaceutical agent delivery and biometric data acquisition device. In some of these embodiments, the pharmaceutical can be vaporized used ultrasonic frequencies that heats and/or cavitates and vaporizes the pharmaceutical agent. In other embodiments, the pharmaceutical agent delivery and biometric data acquisition device vaporizes the pharmaceutical agent by heating it. In exemplary embodiments, the pharmaceutical agent can be heated to between 300° F. and 500° F. in order to vaporize the pharmaceutical agent. In exemplary embodiments, sensory feedback can be given to the user when the pharmaceutical agent delivery and biometric data acquisition device is ready to vaporize the pharmaceutical agent, when the pharmaceutical agent delivery and biometric data acquisition device is vaporizing the pharmaceutical agent, and when the pharmaceutical agent delivery and biometric data acquisition device is finished vaporizing the pharmaceutical agent. In addition, sensory feedback can be given to the user when the vaporized pharmaceutical agent is cleared from the delivery and biometric data acquisition device and safe for storage.

In some embodiments, the user's act of inhaling while the user's mouth is engaged with the mouthpiece of the pharmaceutical agent delivery and biometric data acquisition device activates a timer in the device, which monitors how long the user is inhaling the vaporized pharmaceutical agent. This information can then be recorded in the device's memory and subsequently made available for a third party (e.g., a caregiver or physician). In some embodiments, the length of time in which a user inhales the vaporized pharmaceutical agent corresponds to a particular dose of the vaporized pharmaceutical agent. A user can inhale for about 1 second to about 10 seconds; or about 5 seconds to about 30 seconds; or about 30 seconds to about 1 minute or about 1 minute to about 5 minutes or about 5 minutes and about 15 minutes or about 15 minutes to about 30 minutes; or more depending on the agent inhaled and/or specified parameters for intake.

In some embodiments, the pharmaceutical agent delivery and biometric data acquisition device coupled to the nebulizing unit may be configured to monitor the presence of the liquid pharmaceutical agent before, during, and/or after the nebulization and administration of the pharmaceutical agent. In other embodiments, monitoring for the presence of the liquid pharmaceutical agent corresponds to the time period in which the nebulizing unit is actively vaporizing the liquid pharmaceutical agent. If a dose of the liquid pharmaceutical agent has been fully vaporized, the device will no longer detect the presence of the liquid pharmaceutical agent and the nebulizing unit will be deactivated. If liquid is still present, the device will detect the liquid and continue vaporizing until the liquid has been fully vaporized.

In some embodiments, the pharmaceutical agent delivery and biometric data acquisition device coupled to the nebulizing unit includes a user override function. If during inhalation of the vaporized pharmaceutical agent, a user is interfered with or distracted, the user or other authorized provider can activate an override mechanism which allows the administration of another dose of the vaporized pharmaceutical agent. This information can then be recorded in the device's memory and subsequently made available for a third party (e.g., a caregiver or physician). In some embodiments, however, a supervising caregiver or physician can activate a “kill switch” command, which prevents the user from administering another dose of the vaporized pharmaceutical agent.

In other exemplary embodiments, if a pharmaceutical agent is approved at block 512 method 500 proceeds from block 512 to block 516. At block 516, a dosage of the pharmaceutical agent can be administered via a mouthpiece of the pharmaceutical agent delivery and biometric data acquisition device. In some exemplary embodiments, administering a dosage of the pharmaceutical agent includes, but is not limited to, identifying a pharmaceutical agent associated with the biometric data acquisition device, and determining whether the pharmaceutical agent matches the approved pharmaceutical agent.

In another exemplary embodiment, administering a dosage through a mouthpiece associated with the device can include, but is not limited to, identifying a pharmaceutical agent associated with the biometric data acquisition device; determining whether the pharmaceutical agent matches the approved pharmaceutical agent; commencing administration of the pharmaceutical agent through the mouthpiece of the pharmaceutical agent delivery and biometric data acquisition device, measuring the amount of pharmaceutical agent administered through the mouthpiece of the pharmaceutical agent delivery and biometric data acquisition device; and ceasing administration of the pharmaceutical agent when administration of the predetermined dosage has completed. In accordance with this method, the administration methods can further include repeating this process for all subsequent dosages administered to the subject and include recording biometric data associated with the subsequent dosages. In certain embodiments, the recorded biometric data can be used to evaluate, for example, whether the subject (or healthcare provider administering the pharmaceutical agent) is in compliance with a predetermined treatment plan.

In some embodiments, method 500 may continue to block 518 and where biometric response can be measured related to the dosage administered or the regimen determined for the user. In some embodiments, biometric response of the user can be measured relatively soon or immediately after the pharmaceutical agent has been administered or after a period of time has lapsed. In some embodiments, biometric response of the user can be performed periodically. In other embodiments, the pharmaceutical agent delivery and biometric data acquisition device can be equipped with various sensors to measure one or more of the following biometric responses, for example to evaluate the progress or response of the subject regarding his/her condition: a galvanic skin response, a blood oxygen level response, a body temperature response, a heartrate response, a perfusion index, a blood pressure response, a retina response, an eye movement response, eye color (e.g., yellowing of the sclera), an inhalation velocity response, an inhalation pressure response, an inhalation volume response, an expiratory velocity response, an expiratory pressure response, an expiratory volume response, or an exhale chemical composition response. This list, however, is not inclusive and, therefore, is not meant to be limiting.

In some embodiments, where a biometric response is measured by the pharmaceutical agent delivery and biometric data acquisition device, method 500 can proceed to block 520 where the dosage is updated to generate a revised dosage based on measured biometric response(s). In other embodiments, a pharmaceutical or other monitored agent dose or frequency of administration can be revised by a healthcare professional after the information regarding a previous dosage, the time of a previous dosage, the frequency of a previous dosage and the biological response to a previous dosage or other biometric data has been transmitted and evaluated by the healthcare professional.

In some exemplary embodiments, method 500 may continue to block 522 and record, on the biometric data acquisition device's memory, each time a dosage was administered to a particular user, amount of the dosage, time of day the dosage was administered and date of the dosage administered. In certain aspects, all dosages subsequent to an administered dosage, including revised and unrevised dosages, can be recorded on the device and used to evaluate a user's progress as well as adherence to a pre-determined treatment plan. In some embodiments, this information can be transmitted to an auxiliary electronic device for storage, transport or evaluation etc.

For each of blocks 516, 518, 522, amount of administered dosage, time of the dosage, frequency of the administered dosage, whether or not the dosage was revised, and any other information that may be pertinent in order to monitor treatment of the user can be transmitted to an auxiliary electronic device. The information can be transferred using a wired connection or a wireless connection if and when one becomes available. In some embodiments, until a network connection becomes available, the information can be stored on the pharmaceutical agent delivery and biometric data acquisition device's memory.

FIG. 7 is a flow diagram representing a method 600 illustrating one example of method 500. Method 600 serves as an example and is not meant to be limiting. In some embodiments, user may be visually impaired and any visual cues (e.g., the LED lights) in method 600 can be replaced with other sensory feedback (e.g., auditory or haptic feedback). Method 600 begins by turning on the pharmaceutical agent delivery and biometric data acquisition device at block 601. In some embodiments, this can be done by holding down on a fingerprint reader and pulse oximeter included in the pharmaceutical agent delivery and biometric data acquisition device for a predetermined amount of time. For example, holding down on the fingerprint reader and pulse oximeter for 5 seconds may turn the pharmaceutical agent delivery and biometric data acquisition device on.

Once the pharmaceutical agent delivery and biometric data acquisition device is “on,” method 600 can proceed to block 602 where a fingerprint of a user is scanned by a fingerprint scanner that can be included in the pharmaceutical agent delivery and biometric data acquisition device. At block 604, if the scanned fingerprint does not match a stored fingerprint, then method 600 proceeds to block 605, at which time an indicator, such as a rapidly blinking LED notifies the user that the scanned fingerprint did not match a stored fingerprint. Method 600 then proceeds back to block 602 to allow the user to scan their fingerprint again. If, however, the scanned fingerprint matches a stored fingerprint, then method 600 proceeds to 607, at which time a different indicator, such as a solidly illuminated LED notifies the user that their scanned fingerprint matches a stored fingerprint.

At block 610, method 600 proceeds by determining (e.g. by stored information) whether the user correlated to the scanned and matched fingerprint is approved to take a particular pharmaceutical or other monitored agent. As detailed above, this may include determining the current time and date, when the last time the pharmaceutical agent was administered to the user and whether the current time and date is within an allowable or recommended time period for the user to administer a dose of the pharmaceutical agent.

At block 612, if a determination is made that the user is not approved to take a pharmaceutical agent, then method 600 may proceed to block 613, at which time an indicator such as a rapidly blinking LED or audio notifies the user that the a pharmaceutical agent has not been approved for the user to take at the time of the fingerprint read. If method 600 does proceed to block 613 because the user is not approved to take a pharmaceutical agent, then method 600 can revert back to block 602 or method 600 can end. If, a determination is made that the user being assessed is approved to take a pharmaceutical or other monitored agent at block 612, then method may proceed to block 615, at which time an indicator such as a solidly illuminated LED or audio signal notifies the user that a pharmaceutical agent has been approved to be taken by the user.

After block 615, method 600 proceeds to blocks 616, 617, and/or 619 which can occur concurrently or within a prescribed time period of one another. At block 616, a dosage of the pharmaceutical agent can be administered by the pharmaceutical agent delivery and biometric data acquisition device. The dosage can be administered as describe above in method 500. While the pharmaceutical agent is being administered, and in some embodiments before, an LED or audio can slowly blink or sound-off to notify the user that the pharmaceutical agent is being administered or about to be administered at block 617. When the administration of the pharmaceutical agent is completed, the LED can stop blinking or the audio shuts off Concurrently with the administration of the pharmaceutical agent, the pharmaceutical agent delivery and biometric data acquisition device can be configured to take a picture (e.g. for identification or assessment of after effects etc.) of the user at block 619. In some embodiments, a timestamp can be included with the picture, so that the time that the pharmaceutical agent was administered can be recorded along with a record number and one or more user identifiers (e.g., user's picture).

In another embodiment, method 600 can then proceed to block 622 where the data from administration can be recorded to memory pharmaceutical agent delivery and biometric data acquisition device. In some embodiments, the data recorded can be any of the data discussed above in method 500. Examples include, but are not limited to, pharmaceutical or monitored agent, dose administered of the pharmaceutical agent, time and date of the administration of the pharmaceutical agent, and the biometric response to the administration of the biological agent. In other embodiments, other agents (e.g. over-the-counter agents, vitamins) taken or used by a user can also be recorded by the user using a recorder on the device or other method for recordation in realtime or at a later time by the user.

Method 600 can proceed to block 624 where the recorded data can be transmitted to a secondary electronic device, a cloud computing device or an application stored therein while backed-up by the device. Various user identifiers can be associated with a user's biometric data stored on an electronic record, such that the user can access the biometric data using his/her user identifier. In some embodiments, the user's biometric data is uploaded and stored in a cloud computing device that can be accessed using one or more user identifiers.

A number of variations and modifications of the disclosure can be used. It would be possible to provide for some features of the disclosure without providing others.

For example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure may be dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An agent delivery and biometric data acquisition device comprising:
a housing unit;
a power source;
a processor and memory;
at least one scanner operatively coupled to the processor, wherein the processor verifies an identify of a subject based upon information obtained by the at least one scanner;
an accessory module interface for coupling a nebulizing unit to the device, a nebulizing unit facilitating delivery of one or more agents to the subject;
at least one biometric sensor for sensing biometric data of the subject, wherein the biometric data is sensed and acquired during at least one of prior to, during, and after administering the one or more pharmaceutical agents to the subject, wherein the biometric data is stored in memory; and
at least one data transfer port.

2. The device according to claim 1, wherein the device further comprises a mouthpiece and an image acquisition device centrally aligned with the mouthpiece, the image acquisition device comprising a lens, an image sensor and signal wires which operatively connect the image acquisition device to the processor; and
at least one visual indicator that emits at least one light signal operatively coupled to the image acquisition device and facing the same direction as the lens.

3. The device according to any one of claims 1-2, wherein the at least one biometric sensor includes one or more of a temperature sensor, a thermal imaging sensor, a galvanic skin response sensor, a pulse oximeter, a carbon dioxide sensor, an oxygen sensor, an optical sensor, an air flow velocity sensor, an air pressure sensor, a chemical sensor, and a global positioning system (GPS) sensor.

4. The device according to any one of claims 1-3, wherein the device further comprises one or more additional accessory module interfaces for coupling one or more additional accessory modules to the device, wherein the one or more additional accessory modules is one or more of an injectable syringe, an injectable needle, a syrup dispenser, a spray device, a misting device, and an inhalation mask.

5. The device according to any one of claims 1-4, wherein the device further comprises one or more peripheral module interfaces for coupling one or more peripheral modules to the device.

6. The device according to claim 5, wherein the one or more peripheral modules includes one or more of a blood pressure monitor, a blood glucose monitor, an electrocardiogram device, a battery, and a battery charger.

7. The device according to any one of claims 1-6, wherein the at least one scanner comprises a fingerprint reader.

8. The device according to any one of claims 1-7, wherein the at least one sensor comprises a pressure sensor.

9. The device according to any one of claims 1-8, wherein the nebulizing unit comprises an ultrasonic nebulizing mechanism.

10. The device according to any one of claims 1-9, wherein the nebulizing unit comprises a heat-based nebulizing mechanism.

11. The device according to any one of claims 1-10, wherein the device further comprises an infrared (IR) transmitter and receiver.

12. The device according to any one of claims 1-11, wherein the device further comprises a radio-frequency identification (RFID) reader.

13. The device according to any one of claims 1-12, wherein the device further comprises wireless (e.g.Bluetooth™) hardware and software components.

14. The device according to any one of claims 1-13, wherein the processor further comprises one or more software programs for operating the at least one biometric sensor and for facilitating the acquisition of biometric data using the at least one biometric sensor.

15. The device according to any one of claims 1-14, wherein the one or more agents comprise one or more of albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, a cannabinoid, cannabis oil, tetrahydrocannabinol, tiotropium bromide monohydrate, tobramycin, trimcinolone acetonide, zanamivir, and combinations and derivatives thereof

16. A system for delivering one or more agents to and acquiring biometric data from a subject, the system comprising:
a housing unit;
a power source;
a processor and memory;
at least one scanner operatively coupled to the processor, wherein the processor verifies an identify of the subject based upon information obtained by the at least one scanner;
an accessory module interface for coupling a nebulizing unit to the device, the nebulizing unit facilitating delivery of one or more pharmaceutical agents to the subject;
at least one biometric sensor for sensing the biometric data of the subject, wherein the biometric data is sensed and acquired during at least one of prior to, during, and after administering the one or more agents to the subject, wherein the biometric data is stored in memory; and
at least one data transfer port;
wherein the system facilitates the nebulization and administration of the one or more pharmaceutical agents to the subject, and the acquisition of the biometric data from the subject.

17. The system according to claim 16, wherein the device further comprises a mouthpiece and an image acquisition device centrally aligned with the mouthpiece, the image acquisition device comprising a lens, an image sensor and signal wires which operatively connect the image acquisition device to the processor; and
at least one visual indicator that emits at least one light signal operatively coupled to the image acquisition device and facing the same direction as the lens.

18. The system according to any one of claims 16-17, wherein the at least one biometric sensor includes one or more of a temperature sensor, a thermal imaging sensor, a galvanic skin response sensor, a pulse oximeter, a carbon dioxide sensor, an oxygen sensor, an optical sensor, an air flow velocity sensor, an air pressure sensor, a chemical sensor, and a global positioning system (GPS) sensor.

19. The system according to any one of claims 16-18, wherein the device further comprises one or more additional accessory module interfaces for coupling one or more additional accessory modules to the device, wherein the one or more additional accessory modules is one or more of an injectable syringe, an injectable needle, a syrup dispenser, a spray device, a misting device, and an inhalation mask.

20. The system according to any one of claims 16-19, wherein the system further comprises one or more peripheral module interfaces for coupling one or more peripheral modules to the device.

21. The system according to any one of claims 16-20, wherein the one or more peripheral modules includes one or more of a blood pressure monitor, a glucose monitor, an electrocardiogram device, a battery, and a battery charger.

22. The system according to any one of claims 16-21, wherein the at least one scanner comprises a fingerprint reader.

23. The system according to any one of claims 16-22, wherein the at least one sensor comprises a pressure sensor.

24. The device according to any one of claims 16-23, wherein the nebulizing unit comprises an ultrasonic nebulizing mechanism.

25. The system according to any one of claims 16-24, wherein the nebulizer comprises a heat-based nebulizing mechanism.

26. The system according to any one of claims 16-25, wherein the system further comprises an infrared (IR) transmitter and receiver.

27. The system according to any one of claims 16-26, wherein the system further comprises a radio-frequency identification (RFID) reader.

28. The system according to any one of claims 16-27, wherein the system further comprises wireless (e.g. Bluetooth™) hardware and software components.

29. The system according to any one of claims 16-28, wherein the processor further comprises one or more software programs for operating the at least one biometric sensor and for facilitating the acquisition of biometric data using the at least one biometric sensor.

30. The system according to any one of claims 16-29, wherein the one or more pharmaceutical agents is one or more of albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, a cannabinoid, cannabis oil, tetrahydrocannabinol, tiotropium bromide monohydrate, tobramycin, trimcinolone acetonide, zanamivir, and combinations and derivatives thereof.

31. A method for administering an agent to an authorized user, the method comprising:
scanning a biometric identifier of a user using an agent delivery and biometric data acquisition device;
determining, using the biometric identifier, whether the user is approved to take a agent; and
administering via nebulization a dose of the agent from the agent delivery and biometric data acquisition device, if the user is approved to take the dose of the agent is approved to take the agent.

32. The method according to claim 31, wherein the biometric identifier includes at least one of the following: a fingerprint pattern, an iris pattern, a retina pattern, a vocal pattern, a facial-feature pattern, a pore pattern, a thermal image pattern, or a blood vessel pattern.

33. The method according to any one of claims 31-32, wherein determining whether a user is approved to take the dose of the agent comprises:
matching the scanned biometric identifier to a stored biometric identifier, wherein the stored biometric identifier is an approved user's biometric identifier;
identifying if the agent is included on a list of pharmaceutical agents that are eligible to be taken by the user;
identifying a recent time that the biometric identifier was scanned and the pharmaceutical agent was dispensed; and
approving the administration of the agent if a present time during which the biometric identifier is scanned is within an approved time period for administering the pharmaceutical agent, based on the recent time that the biometric identifier was scanned.

34. The method according to any one of claims 31-33, further comprising recording, on the agent delivery and biometric data acquisition device's memory, times that the agent is administered.

35. The method according to any one of claims 31-34, further comprising transmitting a time that the dosage of the agent was administered, the dosage amount that was administered, and an identity of the agent that was administered to an auxiliary electronic device.

36. The method according to any one of claims 31-35, wherein nebulization of the pharmaceutical agent occurs prior to administering a dosage of the agent to the subject.

37. The method according to any one of claims 31-36, further comprising taking an image of the user, using an imaging device, when administering a dosage of the agent, wherein the imaging device is coupled to the agent delivery and biometric data acquisition device.

38. The method according to any one of claims 31-37, further comprising providing sensory feedback to the user.

39. The method according to claim 38, wherein the sensory feedback is at least one of the following: visual cues, haptic feedback, or auditory feedback.

40. The method according to any one of claims 31-39, further comprising measuring a biometric response to the dose of the agent.

41. The method according to claim 40, wherein the biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response, or an exhale chemical composition response.

42. The method according to any one of claims 40-41, further comprising transmitting the dose of the agent and the measured biometric response to an auxiliary electronic device.

43. The method according to any one of claims 40-42, further comprising administering a revising dosage of the agent based on the measured biometric response.

44. Any of the above claims wherein the agent is a pharmaceutical agent, biological agent or other monitored agent.

45. An agent delivery and biometric data acquisition device comprising:
at least one scanner configured to verify an identify of a subject based upon information obtained by the at least one scanner;
an accessory module interface for coupling a nebulizing unit to the device,
a nebulizing unit coupled to the accessory module interface and configured to facilitate delivery of one or more agents to the subject; and
at least one biometric sensor for sensing biometric data of the subject, wherein the biometric data is sensed and acquired during at least one of prior to, during, and after administering the one or more pharmaceutical agents to the subject.

46. The device according to claim 45, wherein the device further comprises at least one of the following: a housing unit, a power source, a processor, a memory unit and a data transfer port.
